(19) United States
(12) Patent Application Publication
(10) Pub. No.: US 2025/0136349 A1
(43) Pub. Date: May 1, 2025

(54) CONTAINER AND METHOD FOR PRESERVING OR TRANSPORTING BIOLOGICAL MATERIALS

(71) Applicants: United Therapeutics Corporation, Silver Spring, MD (US); Lung Bioengineering Inc., Silver Spring, MD (US)

(72) Inventors: Simone Julie Gregor, Durham, NC (US); Timothy Atolagbe, Hanover, MD (US); Christopher Givens, Chapel Hill, NC (US); Jeffrey Robert SooHoo, Chapel Hill, NC (US); Randolph Vernon Monteiro Shelton, Washington, DC (US)

(73) Assignees: United Therapeutics Corporation, Silver Spring, MD (US); Lung Bioengineering Inc., Silver Spring, MD (US)

(21) Appl. No.: 18/925,364

(22) Filed: Oct. 24, 2024

Related U.S. Application Data
(60) Provisional application No. 63/545,589, filed on Oct. 25, 2023.

(51) Int. Cl. B65D 81/38; B65D 85/50

(52) U.S. Cl. CPC B65D 81/382; B65D 85/50

(57) ABSTRACT

A container for transporting or preserving a biological material includes a bag member that includes a first layer having a peripheral edge and a second layer having a peripheral edge. The peripheral edge of the first layer is adjoined to the peripheral edge of the second layer around a circumference, with a phase change material between the first layer and the second layer. The bag member may further include a sealed first end and an opening defined in a second end. The bag member is configured to receive and hold the biological material.

### RELATED APPLICATIONS

The present application claims priority to U.S. Provisional Patent Application No. 63/545,589 filed Oct. 25, 2023, which is incorporated herein by reference in its entirety.

### FIELD

The present disclosure relates in general to containers and methods for preserving or transporting biological materials and in particular, to containers and methods utilizing a phase change material (PCM) for preserving or transporting biological materials.

### SUMMARY

An embodiment is a container for preserving a biological material, comprising a phase change material enclosed between a first layer and a second layer which form a wall of the container configured to receive and hold the biological material therein.

Another embodiment is a method of preserving a biological material, comprising providing the container of the afore-mentioned embodiment for said biological material, placing said biological material into said container, and preserving the biological material in the container for a period of time, preferably for at least 30 minutes.

Another embodiment is a method of transporting a biological material, comprising providing the container of the afore-mentioned embodiment for said biological material, placing said biological material into said container, and transporting said biological material within said container.

### BRIEF DESCRIPTION OF THE FIGURES

FIGS. 1A-B illustrates a container for a biological material, according to some embodiments.

FIG. 2 illustrates a container for a biological material, according to some embodiments.

FIG. 3 illustrates a container in the form of a bag with a drawstring, according to some embodiments.

FIG. 4A illustrates a container for use with detachable top and/or bottom portions, according to some embodiments.

FIG. 4B illustrates a detachable top and/or bottom portion for use with the container of FIG. 4A, according to some embodiments.

FIGS. 5A-B illustrates different options for the shape of pouches containing a phase change material, according to some embodiments.

FIG. 6 is a chart showing a temperature inside a container over 6 days.

FIGS. 7A and B show graphs of temperature versus elapsed time for organ storage in conventional ice compared to storage in a container according to one embodiment.

FIG. 8 shows a graph of organ storage temperature versus elapsed time in a container according to one embodiment, that was preconditioned using conventional ice.

FIGS. 9A-B show graphs of preservation solution storage temperature in a container holding biological material according to one embodiment where (A) the container was preconditioned and (B) the container was not warmed to room temperature before use.

### DETAILED DESCRIPTION

Unless otherwise specified “a” or “an” means one or more.

As used herein, the term “about” placed before a specific numeric value may mean ±20% of the numeric value; ±18% of the numeric value, ±15% of the numeric value; ±12% of the numeric value; ±8% of the numeric value; ±5% of the numeric value; ±3% of the numeric value; ±2% of the numeric value; ±1% of the numeric value or ±0.5% of the numeric value.

The present disclosure provides a container for transporting or preserving a biological material. In one aspect, preserving the biological material is accomplished based upon the container's ability to maintain an internal temperature within a target temperature range for a desired amount of time, while also maintaining the biological material in a condition suitable for carrying out its intended use after it is contained in the container. Optionally, the container provides a physical barrier to protect the biological material from contact with external surfaces or objects. Preferably, the container's interior is sterile. Optionally, the container has a sealing means to maintain an airtight seal after biological material is placed therein that prevents air from entering or leaving the container. In another aspect, an outer container may be provided into which the container of the biological material is inserted, wherein the outer container provides physical protection and/or an airtight seal around the container of the biological material. In still another aspect, an outer layer of the container provides insulation or constitutes a layer of an insulating material.

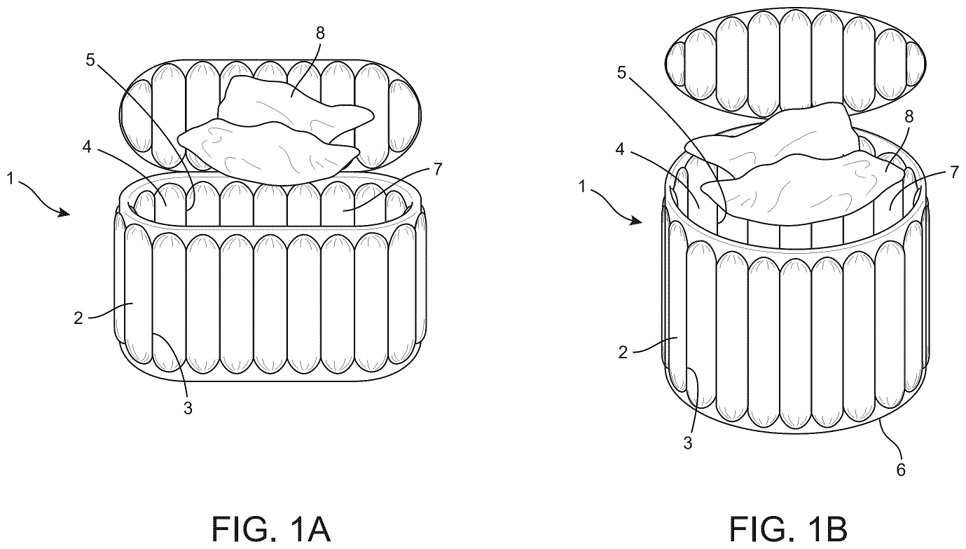

As shown in FIG. 1A, according to one embodiment, the container may include a bag member 1 that includes a first layer 2 having a peripheral edge 3 and a second layer 4 having a peripheral edge 5, the peripheral edge 3 of the first layer 2 adjoined to the peripheral edge 5 of the second layer 4 around a circumference; and a phase change material (not shown) inserted between the first layer 2 and the second layer 4. The bag member 1 further may include a sealed first end 6 and an opening defined in a second end 7. The bag member 1 may be configured to receive and hold the biological material 8. FIG. 1B further illustrates an alternative shape for the bag member 1 of the container of this embodiment.

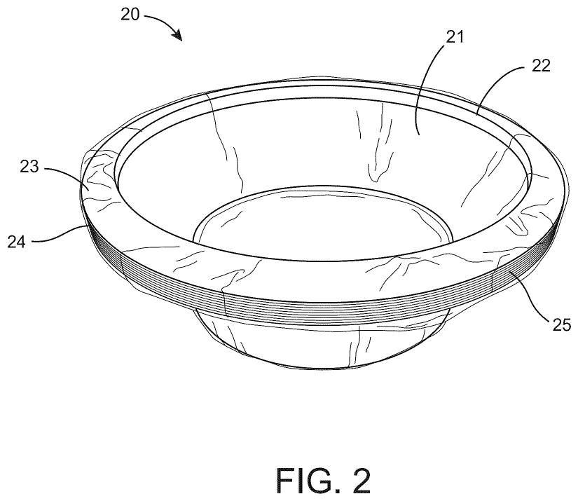

According to another embodiment, the container may include a bowl 20 that includes a first layer 21 having a peripheral edge 22 and a second layer 23 having a peripheral edge 24, the peripheral edge 22 of the first layer 21 adjoined to the peripheral edge 24 of the second layer 23 around a circumference 25; and a phase change material between the first layer 21 and the second layer 23. The bowl 20 may be configured to receive and hold the biological material 8. The container of this embodiment is presented in FIG. 2. In some embodiments, the bowl 20 could be used for receiving (holding, supporting, retaining) a biological material, such as an organ, for example, during a surgery to maintain a desired temperature while preventing overcooling of the biological material.

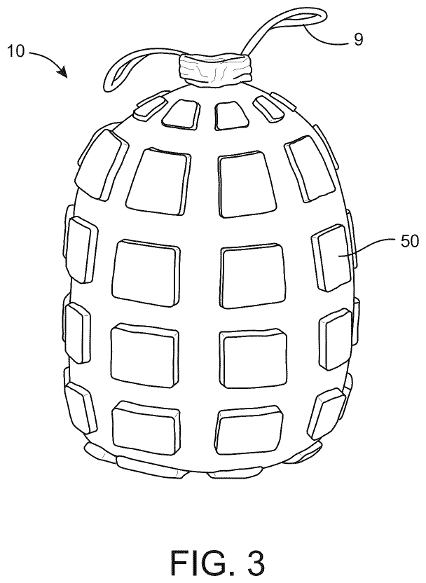

According to another embodiment, as shown in FIG. 3, the container may be in the form of a bag 10, which can optionally be closed by a drawstring 9, or by other suitable means (not shown) such as clasps, magnets, folded-over flap, and the like. In some embodiments, the bag 10 includes sealed pouches, shown as pouches 50, the pouches 50 containing phase change material. In some embodiments, the pouches 50 may decrease in volume as the near the drawstring 9 or other closing mechanism, to facilitate cinching of the bag 10 proximate the drawstring 9. For example, a pouch 50 positioned proximate the drawstring 9 has a volume smaller than that of a pouch 50 positioned in the middle of the bag 10. In some other embodiments, the volume of the pouches 50 is consistent such that any one of the pouches 50 has the same volume of any other one of the pouches 50.

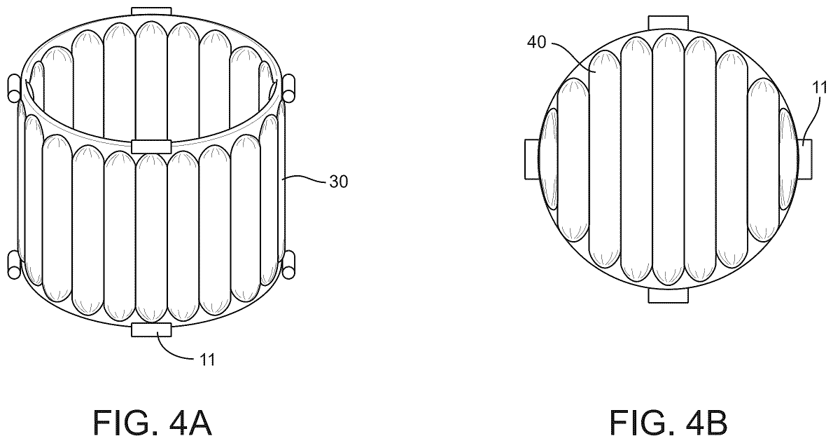

According to another embodiment, as shown in FIGS. 4A-B, the container may have a detachable panel, shown as panel 40, and a center portion, shown as body 30. The panel 40 is configured such that it can be used as a top and/or a bottom of the container. In some embodiments, the container includes two of the panel 40, one for the top of the container, and one for the bottom of the container. The panel 40 and the body 30 each include at least one tab 11. In some embodiments, the panel 40 is configured to selectively and detachably couple with the body 30 via the at least one tabs 11. The tabs 11 may use hook and loop fastener (e.g., Velcro), straps, magnets, or other suitable means for coupling the panel 40 with the body.

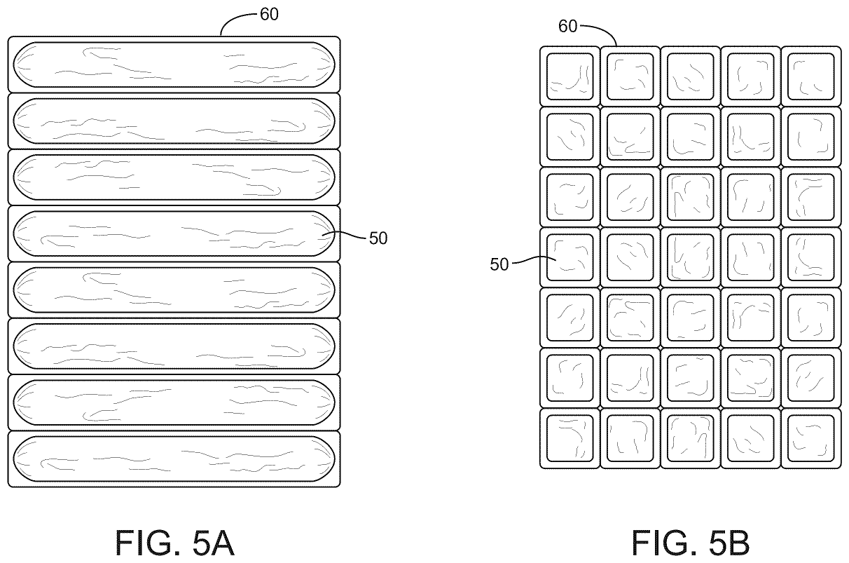

According to another embodiment, as shown in FIGS. 5A-B, phase change sheets 60 can be prepared with the phase change material enclosed within sealed pouches 50. In some embodiments, as shown in FIG. 5A, the pouches 50 may be elliptical in shape, for example arranged parallel to one another and substantially spanning a width of the sheet 60. In some other embodiments, as shown in FIG. 5B, the pouches 50 may be square in shape, for example arranged in an array. In yet other embodiments, the pouches 50 may be any shape. In some embodiments, the phase change sheets 60 include pouches 50, each of the pouches 50 configured to be the same shape. In some other embodiments, the pouches 50 may be various shapes, such that any one of the pouches 50 is elliptical in shape, while any one other of the pouches 50 is square in shape. From the phase change sheets 60, a desired size of the phase change sheet 60 can be cut to suit the design of the container by tearing or cutting between the pouches 50 containing phase change material. The phase change sheets 60 can be used to form body's 30 as in FIG. 4A, and/or other structures, so as to enable custom creation of a container, pouch, cooling surface, etc. as may be suitable for biological materials of different sizes, shapes, etc. For example, the teachings herein may enable intraoperative cutting or tearing of a phase change sheet 60 such that a healthcare provider can form, at the point of care, a container for receiving, maintaining temperature of, and/or transporting an organ or other biological material.

As used herein, the term “biological material” may refer, for example, to an organ, a body fluid, a body tissue, cells or cell cultures, whether naturally occurring or engineered.

The biological material may comprise living cells. In some embodiments, the biological material may be a material of animal origin, i.e., a material originated from an animal. In some embodiments, a biological material may be a material originated from a warm-blooded animal, such as a mammal. For example, the biological material may be a material originated from a primate, such as a human or a monkey, a pig, a dog or a rodent, such as a rat or a mouse.

In some embodiments, the term “organ” refers to a part or structure of the body, which is adapted for a special function or functions. For example, an organ may be a lung, a liver, a kidney, a heart, a pancreas, a bowel, including a stomach and intestines. The organ can be human, animal, 3D printed, or bioengineered.

In some embodiments, a body tissue may be a connective tissue, a muscle tissue, a nervous system tissue or an epithelial tissue from an animal, which may be a warm-blooded animal, such as a mammal. The tissue can be human, animal, 3D printed, or bioengineered.

In some embodiments, a body fluid may be blood, saliva, excreta, a tissue fluid, or a blood component, such as serum or plasma, from an animal, which may be a warm-blooded animal, such as a mammal.

In some embodiments, the biological material may include one or more cells or cell cultures, which may be, for example, reproductive cells, porcine cells, fetal cells, induced pluripotent stem cells (iPS cells) and embryonic stem cells.

The container for preserving a biological material such as an ex vivo organ, e.g. an ex vivo lung may be capable of maintaining a desired preservation temperature inside the container for at least 30 minutes, for at least 1 hour, for at least 2 hours, at least 4 hours, or at least 6 hours, or at least 8 hours, or at least 10 hours, or at least 12 hours, or at least 14 hours, or at least 16 hours, or at least 18 hours, or at least 20 hours, or at least 22 hours, or at least 24 hours. A desired preservation temperature may depend on an application. For example, in some embodiments, a preservation temperature may be from about 0° C. to about 12° C. or from 4° C. to 10° C. or from 6° C. to 10° C. from about 4° C. to about 12° C. or from about 6° C. to about 12° C. or from about 8° C. to about 12° C. or from about 9° C. to about 11° C. or about 10° C. Thus, in some embodiments, the container for preserving a biological material may be capable of maintaining a temperature from about 0° C. to about 12° C. or from about 4° C. to about 12° C. or from about 6° C. to about 12° C. or about 8° C. to about 12° C. or from about 9° C. to about 11° C. or about 10° C. inside the container for at least 30 minutes, for at least 1 hour, for at least 2 hours, at least 4 hours, or at least 6 hours, or at least 8 hours, or at least 10 hours, or at least 12 hours, or at least 14 hours, or at least 16 hours, or at least 18 hours, or at least 20 hours, or at least 22 hours, or at least 24 hours. In some embodiments, a preservation temperature may be from about 4° C. to about 6° C. or about 5° C. Thus, in some embodiments, the container for preserving a biological material, such as an ex vivo organ, e.g. an ex vivo lung, may be capable of maintaining a temperature from about 4° C. to about 6° C. or about 5° C. inside the container for at least 30 minutes, for at least 1 hour, for at least 2 hours, at least 4 hours, or at least 6 hours, or at least 8 hours, or at least 10 hours, or at least 12 hours, or at least 14 hours, or at least 16 hours, or at least 18 hours, or at least 20 hours, or at least 22 hours, or at least 24 hours. In some embodiments, a preservation temperature may be close a human body temperature, such as from about 36° C. to about 38° C. or about 37° C. Thus, in some embodiments, the container for preserving a biological material may be capable of maintaining a temperature from about 36° C. to about 38° C. or about 37° C. inside the container for at least 4 hours, or at least 6 hours, or at least 8 hours, or at least 10 hours, or at least 12 hours, or at least 14 hours, or at least 16 hours, or at least 18 hours, or at least 20 hours, or at least 22 hours, or at least 24 hours.

A container for preserving a biological material may have an internal volume sufficient to accommodate, i.e., to receive and hold, the biological material. For example, the bag member, such as the one in FIGS. 1A-B, may have an internal volume sufficient to accommodate an entirety of a biological material, such as an organ. The bowl, such as the one in FIG. 2, may have an internal volume sufficient to accommodate an entirety of a biological material, such as an organ.

In some embodiments, a container for preserving a biological material may be configured to accommodate, i.e., to receive and hold, a volume of a physiological solution and/or supportive media together with the biological material.

The container for preserving a biological material may also include an outer container, such as an outer transport container, which may be, for example, an organ transport container. The bag member or the bowl may be placed inside the outer container, for example, in a such manner so that wall(s) of the outer container surround the bag member or the bowl having a biological material therein.

The container for preserving a biological material, such as an organ or blood, may be used by placing the biological material inside the container, e.g. inside the inner volume of the bag member or the bowl, and preserving the biological material at a desired preservation temperature provided by the PCM for at least 30 minutes, for at least 1 hour, for at least 2 hours, for at least 4 hours, or at least 6 hours, or at least 8 hours, or at least 10 hours, or at least 12 hours, or at least 14 hours, or at least 16 hours, or at least 18 hours, or at least 20 hours, or at least 22 hours, or at least 24 hours. A preferred time range is from 30 minutes to 3 hours.

In some embodiments, a biological material. such as an organ, blood or a cell culture, may be placed into the container without initially precooling the container, i.e. lowering the temperature inside the container below a surrounding or room temperature, such as 25° C. or 20° C. Yet in some other embodiments, a biological material. such as an organ, blood or a cell culture, may be placed into the container after initially precooling the container, i.e. lowering the temperature inside the container below a surrounding or room temperature, such as 25° C. or 20° C., by at least 2° C., at least 3° C., at least 4° C., at least 5° C., at least 6° C. or at least 7° C.

The container for preserving a biological material. such as an organ, blood or a cell culture, may be used for transporting the biological material by placing the biological material inside the container, e.g. inside the inner volume of the bag member or the bowl, and transporting the biological material within the container at a desired preservation temperature.

After being preserved and/or transported with the container, the biological material, such as an organ, which may be, for example, a lung, a liver, a kidney, a heart, a pancreas, a bowel, including a stomach and intestines, may be transplanted into a subject, such as a warm-blooded animal, e.g., a mammal, such as a human being.

Containers for preserving a biological material of this disclosure may significantly reduce costs associated with preserving, storing and/or transporting biological materials. In addition, the container is able to maintain the biological material during the period of time in which it is contained in the container in a state that is suitable for its intended use, e.g., it maintains a kidney in a condition suitable for performing a transplant into a recipient after the period of time in which it is contained in the container.

### Phase Change Material

A phase-change material (PCM) is a substance which releases/absorbs sufficient energy at phase transition to provide useful heat or cooling. In some embodiments, a phase transition in PCM occurs from one of the two fundamental states of matter—solid and liquid—to the other. Yet in other embodiments, a phase transition in PCM may occur between non-classical states of matter, such as between two crystalline structure states, with one crystalline structure conforming to another, which may be a higher or lower energy state.

In some embodiments, PCM may be an organic PCM. Non-limiting examples of organic PCM include hydrocarbons, such as paraffins (CₙH₂ₙ₊₂), lipids and sugar alcohols.

In some embodiments, an organic PCM may be an organic PCM disclosed in U.S. Pat. Nos. 10,703,950 and 11,655,408; and US20200108367, each of which is incorporated herein by reference in its entirety. In some embodiments, an organic PCM may be in a composition, such as one of compositions in U.S. Pat. Nos. 10,703,950 and 11,655,408; and US20200108367.

An organic PCM may be polymeric. Yet in some embodiments, an organic PCM may be non-polymeric. In some embodiments, an organic PCM may not comprise a hydrocarbon. In some embodiments, an organic PCM may not comprise an alkane. In some embodiment, an organic PCM may not comprise octadecane.

In some embodiments, an organic PCM may be selected from the group consisting of aliphatic hydrocarbons, halogenated hydrocarbons, waxes, fats, mono- di- and tri-glycerides, fatty alcohols, fatty acids, fatty amines, fatty amides, amino acids, amine salts, urethanes, sarcosinates, sugars, sugar alcohols, alcohols, polyesters, ethers, aldehydes, ketones and esters, salts and mixtures thereof.

In some embodiments, an organic PCM may be selected from waxes, fats, mono- di- and tri-glycerides, fatty alcohols, fatty acids, fatty amines, fatty amides, amino acids, amine salts, urethanes, sarcosinates, sugars and sugar alcohols, and esters, salts and mixtures thereof.

In some embodiments, an organic PCM may be selected from fatty acids, fatty alcohols, fatty amines and derivatives thereof, preferably fatty acids, fatty alcohols and derivatives thereof, particularly fatty acids and derivatives thereof. Suitable derivatives may be esters, amides or salts, preferably esters or salts, particularly esters.

In some embodiments, an organic PCM may be selected from fatty acids, fatty alcohols and fatty amines, and esters, amides or salts thereof.

In some embodiments, an organic PCM may be an ester or a diester. In some embodiments, an organic PCM may comprise an ester. In some embodiments, an organic PCM may consist of an ester. In some embodiments, an organic PCM may comprise a mixture of a first ester and a second ester. In some embodiments, a PCM ester may comprise a linear alcohol. In some embodiments, a PCM ester may comprise a linear carboxylic acid. In some embodiments, a PCM ester may comprise a linear alcohol and a linear carboxylic acid. In some embodiments, a PCM ester may comprise a mono-alcohol. In some embodiments, a PCM ester may comprise a mono-carboxylic acid. In some embodiments, a PCM ester may comprise a mono-alcohol and a mono-carboxylic acid. In some embodiments, an organic PCM may comprise a fatty acid ester or a fatty alcohol ester. In some embodiments, an organic PCM may consist of a fatty acid ester or a mixture of fatty acid esters.

In some embodiments, an organic PCM may be selected from the group consisting of methyl decanoate, methyl undecanoate, methyl laurate, methyl myristate, methyl palmitate, methyl stearate, methyl arachidate, methyl behenate, ethyl decanoate, ethyl undecanoate, ethyl laurate, ethyl myristate, ethyl palmitate, ethyl stearate, ethyl arachidate, ethyl behenate and mixtures thereof.

In some embodiments, an organic PCM may be selected from the group consisting of propyl decanoate, propyl undecanoate, propyl laurate, propyl myristate, propyl palmitate, propyl stearate, propyl arachidate, propyl behenate, butyl decanoate, butyl undecanoate, butyl laurate, butyl myristate, butyl palmitate, butyl stearate, butyl arachidate, butyl behenate and mixtures thereof.

In some embodiments, an organic PCM may be selected from the group consisting of pentyl decanoate, pentyl undecanoate, pentyl laurate, pentyl myristate, pentyl palmitate, pentyl stearate, pentyl arachidate, pentyl behenate, hexyl decanoate, hexyl undecanoate, hexyl laurate, hexyl myristate, hexyl palmitate, hexyl stearate, hexyl arachidate, hexyl behenate and mixtures thereof.

In some embodiments, an organic PCM may be selected from the group consisting of heptyl octanoate, heptyl pelargonate, heptyl decanoate, heptyl undecanoate, heptyl laurate, heptyl myristate, heptyl palmitate, heptyl stearate, heptyl arachidate, heptyl behenate, octyl heptanoate, octyl octanoate, octyl pelargonate, octyl decanoate, octyl undecanoate, octyl laurate, octyl myristate, octyl palmitate, octyl stearate, octyl arachidate, octyl behenate and mixtures thereof.

In some embodiments, an organic PCM is selected from the group consisting of nonyl propionate, nonyl butanoate, nonyl pentanoate, nonyl hexanoate, nonyl heptanoate, nonyl octanoate, nonyl pelargonate, nonyl decanoate, nonyl undecanoate, nonyl laurate, nonyl myristate, nonyl palmitate, nonyl stearate, nonyl arachidate, nonyl behenate, decyl acetate, decyl propionate, decyl butanoate, decyl pentanoate, decyl hexanoate, decyl heptanoate, decyl octanoate, decyl pelargonate, decyl decanoate, decyl undecanoate, decyl laurate, decyl myristate, decyl palmitate, decyl stearate, decyl arachidate, decyl behenate and mixtures thereof.

In some embodiments, an organic PCM may be selected from the group consisting of lauryl formate, lauryl acetate, lauryl propionate, lauryl butanoate, lauryl pentanoate, lauryl hexanoate, lauryl heptanoate, lauryl octanoate, lauryl pelargonate, lauryl decanoate, lauryl undecanoate, lauryl laurate, lauryl myristate, lauryl palmitate, lauryl stearate, lauryl arachidate, lauryl behenate and mixtures thereof.

In some embodiments, an organic PCM may be selected from the group consisting of myristyl formate, myristyl acetate, myristyl propionate, myristyl butanoate, myristyl pentanoate, myristyl hexanoate, myristyl heptanoate, myristyl octanoate, myristyl pelargonate, myristyl decanoate, myristyl undecanoate, myristyl laurate, myristyl myristate, myristyl palmitate, myristyl stearate, myristyl arachidate, myristyl behenate and mixtures thereof.

In some embodiments, an organic PCM may be selected from the group consisting of cetyl formate, cetyl acetate, cetyl propionate, cetyl butanoate, cetyl pentanoate, cetyl hexanoate, cetyl heptanoate, cetyl octanoate, cetyl pelargonate, cetyl decanoate, cetyl undecanoate, cetyl laurate, cetyl myristate, cetyl palmitate, cetyl stearate, cetyl arachidate, cetyl behenate and mixtures thereof.

In some embodiments, an organic PCM may be selected from the group consisting of butyl decanoate, methyl laurate, octyl laurate, lauryl laurate, lauryl pelargonate, octyl myristate, myristyl pelargonate, nonyl laurate, methyl myristate, decyl laurate, octyl palmitate, lauryl caprate, cetyl octanoate, methyl palmitate, methyl stearate, lauryl laurate, octyl stearate, decyl palmitate, stearyl pelargonate, lauryl myristate, decyl stearate, stearyl caprate and cetyl palmitate. Preferably, the PCM is selected from the group consisting of decyl laurate, methyl stearate, methyl palmitate, lauryl laurate and cetyl palmitate.

In some embodiments, an organic PCM may be selected from the group consisting of stearyl formate, stearyl acetate, stearyl propionate, stearyl butanoate, stearyl pentanoate, stearyl hexanoate, stearyl heptanoate, stearyl octanoate, stearyl pelargonate, stearyl decanoate, stearyl undecanoate, stearyl laurate, stearyl myristate, stearyl palmitate, stearyl stearate, stearyl arachidate, stearyl behenate and mixtures thereof.

In some embodiments, PCM may be an inorganic PCM, such as a salt hydrate, which may be a metal inorganic salt with water molecule(s) of hydration. In some embodiments, an inorganic PCM may have, for example, a formula (MₓN_{y}·nH₂O), where n is an positive integer, such as 1, 2, 3 etc. Non-limiting examples of inorganic PCM include Glauber's salt (sodium sulfate decahydrate), calcium chloride hexahydrate and sodium carbonate.

In some embodiments, a PCM may be in a composition, which also includes one or more additional components, which may be selected from the group consisting of nucleating agents, thermal stabilizers, anti-oxidants, fire retardants, structuring agents, binders, inorganic particles, such as metals or metal oxides like silica, carbon particles and mixtures thereof.

A nucleating agent may be used to prevent sub-cooling of the PCM composition. In some embodiments, a nucleating agent may be selected from a fatty acid, a fatty alcohol, a fatty amide, a paraffin, a polyether and mixtures thereof. In some embodiments, a nucleating agent may be a wax. In some embodiments, a nucleating agent may be selected from squalane wax, behenyl behenate, stearic acid, lauric acid, myristic acid, palmitic acid, behenic acid, stearyl alcohol, stearamide, beeswax, montane wax, dicalite, graphite, fumed silica, precipitated silica, potassium dihydrogen phosphate, calcium sulfate and mixtures thereof.

A thermal stabiliser may be used to preventing or retard thermally induced decomposition or isomerization of the PCM composition. For example, a thermal stabilizer may prevent or retard formation of lower molecular weight products or isomers resulting from thermally induced decomposition or isomerization of the ester. The thermal stabiliser may be selected from phosphites, phosphonites, phosphate esters and mixtures thereof.

An anti-oxidant may be used to prevent or retard oxidation of the PCM composition. For example, an antioxidant may prevent or retard formation of products resulting from reaction of the ester with atmospheric oxygen or with oxygen free radicals, which products may include, for example, alcohols, aldehydes, acids, peroxides, or water. The anti-oxidant may be selected from phenolic antioxidants, sterically hindered phenolic antioxidants, thioether antioxidants, and mixtures thereof.

A fire retardant may be used for fire safety purposes or to conform with fire safety regulations for some uses of the PCM composition. The fire retardant may be selected from a halogenated hydrocarbon, a phosphate ester and mixtures thereof. The fire retardant may be selected from chloroparaffin, bromooctadecane, bromopentadecane, bromononadecane, bromoeicosane, bromodocosane and mixtures thereof. Other possible flame retardants include bis(pentabromophenyl) oxide or bis(tetrabromophenyl) oxide.

A structuring agent may be used to assist in the containment of the PCM composition. For example, a PCM containing composition may change from solid to liquid and vice versa many times during use, and the structuring agent may add structure the liquid PCM so that it is easier to contain. In some embodiments, structuring agent may be a gellant. In some embodiments, a structuring agent may be selected from structuring polymers, gelling polymers, thixotropic polymers and mixtures thereof. In some embodiments, a structuring agent may be selected from polyamides, polyurethanes, polyethers, polyacrylates and copolymers and mixtures thereof.

In some embodiments, a PCM or a PCM containing composition may have a melting point of at least about −10° C., or at least about −5° C., or at least about 0° C., or at least about 5° C. For example, in some embodiments, a PCM or a PCM containing composition may have a melting point of about 9.5° C. In some embodiments, a PCM or a PCM containing composition may have a melting point of at most 50° C., or at most 40° C., or at most 30° C., or at most 25°, or at most 20° C. For example, a PCM or a PCM containing composition may have a melting point in the range from about −10° C. to about 50° C., or from about −5° C. to about 40° C. or from about 0° C. to about 30° C. or from about 5° C. to about 20° C. or any value within those ranges. A melting point may be measured, for example, by Differential Scanning calorimetry (DSC).

In some embodiments, a PCM or a PCM containing composition may have a latent heat of fusion (i.e., the latent heat of the transition from solid to liquid) of at least 100 J/g, preferably at least 150 J/g, more preferably at least 160 J/g, yet more preferably at least 170 J/g. In some, embodiments, a PCM or a PCM containing composition may have a latent heat of fusion of at most 300 J/g, preferably at most 250 J/g, more preferably at most 230 J/g, yet more preferably at most 210 J/g. In some embodiments, a PCM or a PCM containing composition may have a latent heat of fusion in the range from 100 to 300 J/g, or from 100 to 250 J/g or from 160 to 230 J/g or from 170 to 210 J/g. The latent heat of fusion may be measured, for example, by DSC.

In some embodiments, a PCM or a PCM containing composition may have a crystallization temperature of at least about −15° C., or at least about −10° C., or at least about −5° C., or at least about 0° C. In some embodiments, a PCM composition or a PCM containing composition may have a crystallization temperature of at most about 45° C., or at most about 35° C., or at most about 30° C., or at most about 25° C. In some embodiments, a PCM or a PCM containing composition has a crystallization temperature in the range from about −15° C. to about 45° C., or from about −10° C. to about 35° C. or from about −5° C. to about 25° C. or from 0° C. to 25° C. The crystallization temperature may be measured, for example, by DSC.

In some embodiments, a difference between a melting point and a crystallization temperature of a PCM or a PCM containing composition may be less than 10° C., or less than 9° C., or less than 8° C., or less than 7° C., or less than 6° C., or less than 5° C., or less than 4° C.

In some embodiments, a PCM containing composition may contain a PCM in the amount of at least 75 weight (wt) %, or at least 80 wt %, or at least 85 wt %, or at least 90 wt %, or at least 95 wt %. In some embodiments, a PCM containing composition may contain a PCM in the amount of at most 99.99 wt % or at most 99.9 wt %, or at most 99 wt %, or at most 95 wt %.

In some embodiments, a PCM or a PCM containing composition may have a melting temperature of about 9.5° C. In some embodiments, a PCM or a PCM containing composition may have a crystallization temperature of about 6° C. In some embodiments, a PCM or a PCM containing composition may have a melting temperature of about 9.5° C. and a crystallization temperature of about 6° C.

PCM containing compositions are commercially available, for example, from Croda, Inc. and PCM products, Ltd. Non-limiting examples of commercially available PCM containing compositions include CrodaTherm™ 5; CrodaTherm™ 6.5; CrodaTherm™ 9.5; CrodaTherm™ 19; CrodaTherm™ 21; CrodaTherm™ 24W; CrodaTherm 37. For example, CrodaTherm™ 9.5 is capable of maintaining a temperature of about 10° C. for at least 24 hours.

Embodiments described herein are further illustrated by, though in no way limited to, the following working examples.

### Example

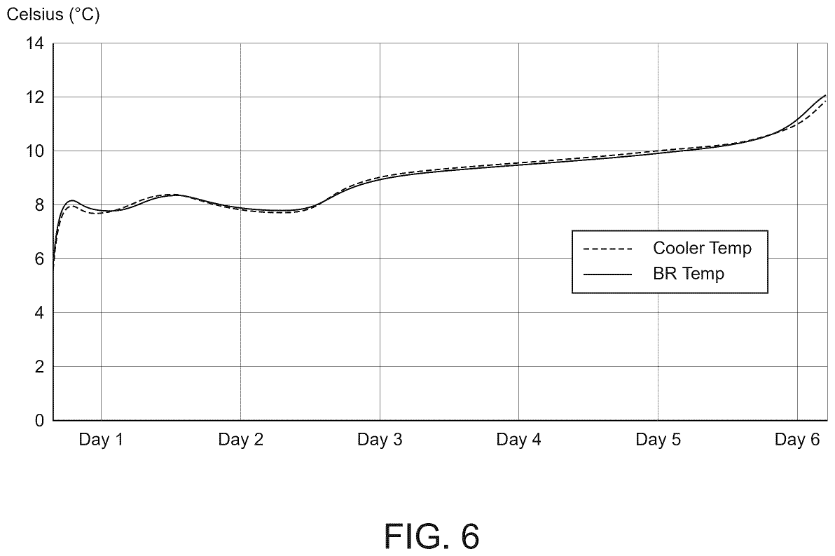

FIG. 6: Temperature stability over extended storage of preservation solution in a PCM container. 4 L of preservation solution was chilled to 4 C and placed inside a PCM container. This container was placed in a Styrofoam box and the void space was filled with conventional ice. Temperature was monitored between the bag of preservation solution and the PCM container (blue) as well as inside the preservation solution bag (red). Temperature was monitored for several days.

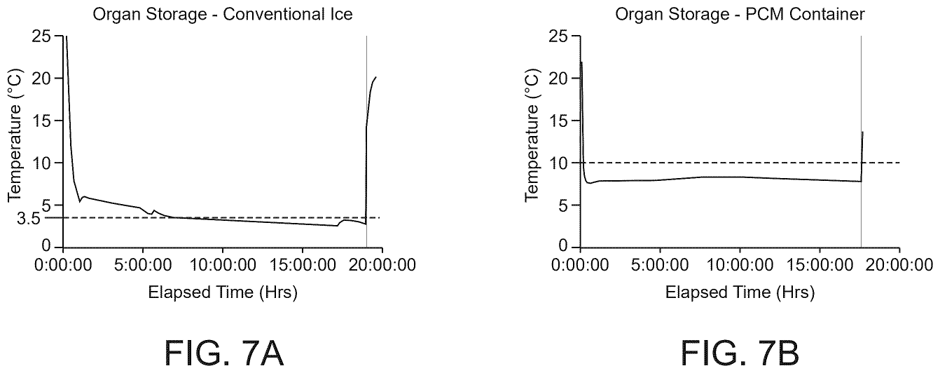

FIGS. 7A-B: Organ storage in conventional ice compared to lung storage in a PCM container. (A) An organ was cold preserved and then placed in a bag containing 4 C preservation solution. This was then placed in a cooler and the void space was filled with conventional ice. The temperature was recorded over about 20 hours of storage. (B) An organ was cold preserved and then placed in a bag containing 4 C preservation solution. This was then placed inside a PCM container. The PCM container was then placed in a cooler, the void space was filled with conventional ice, and the organ was stored for about 20 hours. In both graphs, the tan line represents organ removal from storage.

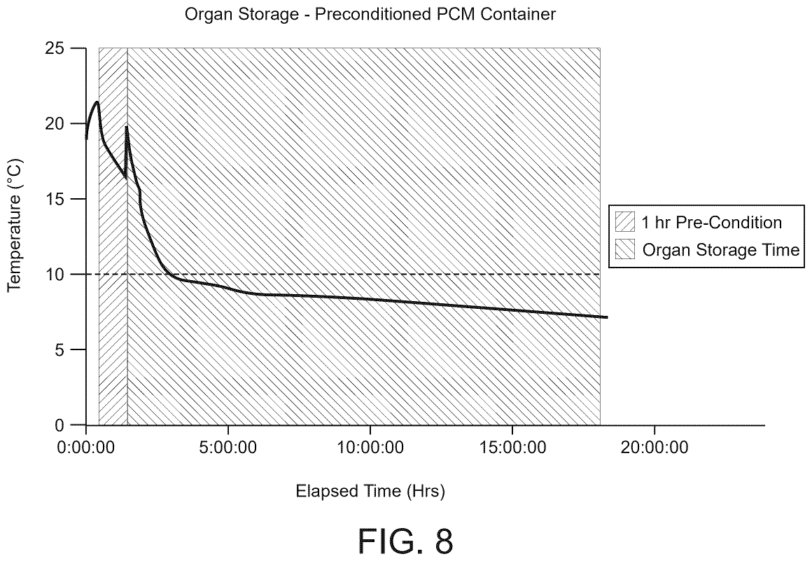

FIG. 8: Organ storage in a preconditioned PCM Container. A PCM container was placed in a cooler and the void space was filled with conventional ice. This was held for 1 hour, after which a cold preserved organ in a bag of 4 C preservation solution was placed inside the PCM container. The temperature was then monitored for about 20 hours.

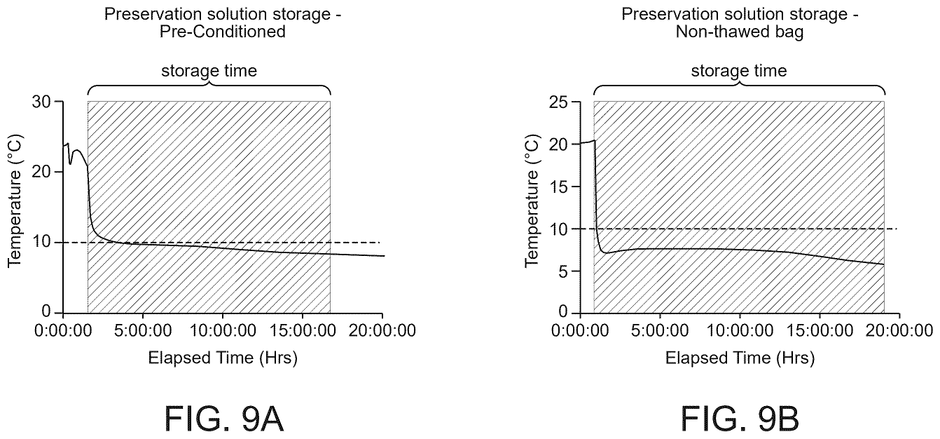

FIGS. 9A-B: Preservation solution storage in PCM containers. (A) A PCM container was pre-conditioned by placing it in a cooler and filling the void space with conventional ice. A bag of 4 C preservation solution was then placed into the pre-conditioned PCM container and the temperature was monitored for about 20 hours. (B) A PCM container was not fully warmed to room temperature before placing a bag of 4 C preservation solution inside the PCM container, and storing in a cooler where the void space was filled by conventional ice. The tan boxes indicate storage time.

Although the foregoing refers to particular preferred embodiments, it will be understood that the present invention is not so limited. It will occur to those of ordinary skill in the art that various modifications may be made to the disclosed embodiments and that such modifications are intended to be within the scope of the present invention.

All of the publications, patent applications and patents cited in this specification are incorporated herein by reference in their entirety.

What is claimed is:

1. A container for preserving a biological material, comprising a phase change material enclosed between a first layer and a second layer which form a wall of the container configured to receive and hold the biological material therein.

2. The container of claim 1, further comprising an outer transport container wall.

3. A container for preserving a biological material, comprising:
a bowl comprising a first layer having a peripheral edge and a second layer having a peripheral edge, the peripheral edge of the first layer adjoined to the peripheral edge of the second layer around a circumference; wherein said bowl is configured to receive and hold the biological material therein; and
a phase change material between the first layer and the second layer.

4. The container of claim 1, wherein the phase change material is an organic phase change material (PCM).

5. The container of claim 4, wherein the phase change material is octyl laurate.

6. The container of claim 1, wherein the phase change material is an inorganic phase change material.

7. The container of claim 6, wherein the inorganic phase change material is a salt hydrate.

8. The container of claim 1, wherein the container is capable of maintaining a temperature of at least about 8° C. to about 12° C. for at least 8 hours.

9. The container of claim 1, wherein the container is capable of maintaining a temperature of about 10° C. for at least 8 hours.

10. The container of claim 1, wherein the phase change material has a melting point of about 9.5° C.

11. The container of claim 1, wherein the biological material comprises an organ, a body fluid, a body tissue, cells or cell cultures.

12. The container of claim 1, wherein the biological material is an organ selected from the group consisting a lung, a heart, a kidney, a liver, a pancreas and a small intestine.

13. The container of claim 11, wherein the biological material comprises blood.

14. The container of claim 12, wherein the biological material is a lung.

15. The container of claim 11, wherein the biological material comprises one or more cells or cell cultures selected from the group consisting of reproductive cells, porcine cells, fetal cells, induced pluripotent stem cells (iPS cells) and embryonic stem cells.

16. The container of claim 1, wherein the bag member or the bowl is further configured to receive and hold a volume of a physiological solution or supportive media therein.

17. A method of preserving a biological material, comprising providing the container of claim 1 for said biological material, placing said biological material into said container, and preserving the biological material in the container for a period of time.

18. A method of transporting a biological material, comprising providing the container of claim 1 for said biological material, placing said biological material into said container, and transporting said biological material within said container.

19. The method of claim 17, wherein the biological material organ is an ex vivo organ.

20. The method of claim 19, wherein the organ is a lung.

21. The method of claim 17, wherein the container preserves a temperature of the biological material at a temperature of 0° C. to 12° C. for at least 5 hours.

22. The method of claim 17, wherein the container preserves a temperature of the biological material at a temperature of 0° C. to 12° C. for at least 10 hours.

23. The method of claim 17, wherein the container preserves a temperature of the biological material at a temperature of 0° C. to 12° C. for at least 15 hours.

24. The method of claim 17, wherein the container preserves a temperature of the biological material at a temperature of 0° C. to 12° C. for at least 20 hours.

25. The method of claim 17, wherein the method does not comprise cooling the container prior to the placing said biological material into said container.

26. The method of claim 17, wherein the method comprises cooling the container prior to the placing said biological material into said container.
